(19) United States
(12) Patent Application Publication
(10) Pub. No.: US 2020/0261622 A1
(43) Pub. Date: Aug. 20, 2020

(54) COMPOSITES FOR LOAD-BEARING APPLICATIONS

(71) Applicant: University of Connecticut, Farmington, CT (US)

(72) Inventors: Bryant Heimbach, Stamford, CT (US); Mei Wei, Athens, OH (US)

(21) Appl. No.: 16/794,655

(22) Filed: Feb. 19, 2020

Related U.S. Application Data
(60) Provisional application No. 62/807,468, filed on Feb. 19, 2019.

(51) Int. Cl. A61L 27/44; A61L 27/36; C08L 67/04; C08J 5/04; A61L 27/12

(52) U.S. Cl. CPC A61L 27/446; A61L 27/365; C08L 67/04; A61L 2400/12; C08J 5/046; A61L 27/12; C08J 5/045

(57) ABSTRACT

A load-bearing bone fixation composite includes a polymer matrix, a plurality of polymer fibers aligned along a common axis and disposed in the polymer matrix, wherein the polymer matrix binds the surface of the polymer fibers, and a plurality of high aspect ratio nanorods coating at least a portion of each of the polymer fibers, wherein the long axis of at least a portion of the nanorods is aligned with the common axis, and wherein the high aspect nanorods have an aspect ratio of 10 or greater. Further included is a bone fixation device including the foregoing composite. A method of bone fixation comprises affixing the foregoing composite to a site of a load-bearing bone fracture, or maxillofacial bone fracture. Also included are methods of making the composites.

### CROSS-REFERENCE TO RELATED APPLICATIONS

This application claims priority to U.S. Provisional Application 62/807,468 filed on Feb. 19, 2019, which is incorporated herein by reference in its entirety.

### STATEMENT REGARDING FEDERALLY SPONSORED RESEARCH & DEVELOPMENT

This invention was made with government support under IIP-1414274 awarded by the National Science Foundation. The government has certain rights in the invention.

### FIELD OF THE DISCLOSURE

The present disclosure is related to bioresorbable composite materials having high strength and stiffness for bone fixation applications, for example.

### BACKGROUND

Investment in the biomaterials industry has been rapidly growing in recent years. For example, the market for implantable biomaterials is projected to generate $11.9 billion in revenue by the year 2019. In addition, the most common orthopedic-related trauma cases are bone fractures, which often require a fixation device to help heal the bone properly. Currently, metals are considered the clinical standard for bone fixation devices, however, there are many undesirable effects associated with using metal for fixation in vivo, including stress shielding, metal ion leaching, and poor integration between bone and metal implant. Stress shielding stems from the use of fixation materials that are significantly stiffer than natural bone (i.e., metals have an elastic modulus of 110-210 GPa, while natural bone has a modulus ranging from about 8-25 GPa), which results in the load being imparted on the device rather than the bone, and subsequently results in a localized decrease in bone mineral density. Meanwhile, metal ion leaching increases inflammation and irritation around the implant. Due to both effects, there is often a need for a second surgery to remove the fixation device, leading to higher medical costs and greatly increased patient discomfort. For these reasons, there has been great interest in making a fixation device that is mechanically sound enough to properly support the healing of bone, while being fully degradable to eliminate the need for a second surgery.

There have been many researched materials that are able to safely degrade in vivo, but their mechanical properties typically fall short of what is required for a load-bearing fracture. As such, composite materials have been investigated to provide better mechanical support than degradable polymers alone. An example of such a composite includes polylactic acid (PLA) with hydroxyapatite particle reinforcement. The composite formed by an in situ-precipitation method has a Young's modulus of 3.6 GPa and a strength of 155 MPa, which is neither strong nor stiff enough for load-bearing applications. The best results thus far involve the use of a polymer matrix reinforced with bioceramic particles. Granules of PLLA with uniformly-distributed HA microparticles were hot compression molded to make HA-reinforced PLLA composites. The resulting composite bars had a bending modulus and strength of 10 GPa and 270 MPa, respectively. Despite showing the best properties for such a composite, the composites still left much to be desired with regards to bending stiffness for use as a load-bearing implant. To overcome the relatively poor mechanical properties of polymer-based degradable materials, degradable metals, such as magnesium, have been investigated as well. Such materials have been shown to have mechanical properties similar to bone, however, magnesium has been shown to release hydrogen gas during in vivo degradation and causes localized inflammation, indicating the need for further improvements to make viable degradable fixation devices. Most recently, a composite containing both biodegradable long-fiber reinforcement and particle reinforcement was made. With this formulation, the composite material achieved a bending modulus of 9.2 GPa and a bending strength of 187 MPa while showing remarkable toughness.

What is needed are novel composites that have high strength and stiffness for bone fixation applications.

### BRIEF SUMMARY

In one aspect, a load-bearing bone fixation composite comprises a polymer matrix; a plurality of polymer fibers aligned along a common axis and disposed in the polymer matrix, wherein the polymer matrix binds the surface of the polymer fibers; and a plurality of high aspect ratio nanorods coating at least a portion of each of the polymer fibers, wherein the long axis of at least a portion of the nanorods is aligned with the common axis, and wherein the high aspect nanorods have an aspect ratio of 10 or greater.

In another aspect, a bone fixation device comprises the foregoing composite.

In yet another aspect, a method of bone fixation comprises affixing the foregoing composite to a site of a load-bearing bone fracture, or maxillofacial bone fracture.

In a still further aspect, a method of making a load-bearing bone fixation composite comprises coating a plurality of polymer fibers with a suspension containing a polymer matrix, a plurality of high aspect ratio nanorods, and a solvent for the polymer matrix, wherein the polymer matrix binds the surface of the polymer fibers, and wherein the high aspect nanorods have an aspect ratio of 10 or greater; optionally repeating the coating; aligning the plurality of coated polymer fibers; and consolidating the plurality of coated polymer fibers to provide the composite.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic of a composite according to the present disclosure.

FIG. 2 shows an overview of the processing techniques used to fabricate the composite samples described herein.

FIG. 3 shows the individual fibrils of the silk fibroin (3a) and poly-1-lactic acid (3b) reinforcement fibers.

FIG. 4 shows FTIR spectra showing the presence of CTAB on the surface of the HA nanoparticles (4a) and a TEM image of the HA nanoparticles used for the particle reinforcement phase for this study (4b).

FIG. 5 shows the effect of long fiber reinforcement type (5a), the effect of HA amount (5b), and the effect of the two-way interaction of matrix type and amount (5c) on flexural modulus.

FIG. 6 shows the effect of long-fiber reinforcement type (6a), matrix type (6b), matrix amount (6c), HA amount (6d), and the two-way interaction of matrix amount and HA amount (6e) on flexural strength.

FIG. 7 shows the effect of long-fiber reinforcement type (7a), matrix type (7b), matrix amount (7c), HA amount (7d), and the two-way interactions of HA amount and fiber type (7d), HA amount and matrix type (7e), and HA amount and matrix amount (7f) on the toughness of the composite samples.

FIG. 8 shows the observed versus predicted values according to the DOE model analyzing the effect of previously-mentioned factors on bending modulus (8a), strength (8b), and toughness (8c).

FIG. 9 shows the error present on each run of the DOE for bending modulus (9a), strength (9b), and toughness (9c), showing consistency throughout the study (i.e. error present was random and not influenced by controllable outside forces).

FIG. 10 is a plot of flexural modulus and strength (10a) and flexural toughness (10b) of the SF/HA reinforced PLA composites with respect to HA vol % within the composite.

FIG. 11 shows the stress vs. strain curves for composite samples with 0, 16, and 24 vol % HA, where the samples with 0 and 24 vol % HA show delamination with negligible plastic deformation and the sample with 16 vol % HA exhibits plastic deformation and significantly higher strength and toughness compared to other formulations of the composite.

FIG. 12 is FESEM images showing the transverse (12a) and longitudinal (12b) cross sections of a composite sample containing 16 vol % HA.

FIG. 13 shows XRD spectra confirming HA was synthesized and that the acetic acid wash did not cause any phase changes (13a) and FTIR spectra (13b) showing that the acetic acid wash was effective at removing the gelatin coating on the HA.

FIG. 14 shows FESEM images of the HA nanowhiskers before (14a) and after (14b) the acetic acid wash.

FIG. 15 shows flexural stress versus strain curves for the rectangular composites.

FIG. 16 shows results from the cell proliferation alamarBlue™ assay (16a) and the cell viability blue/green assay (16b), where dim dots represent all cells and bright dots represent dead cells.

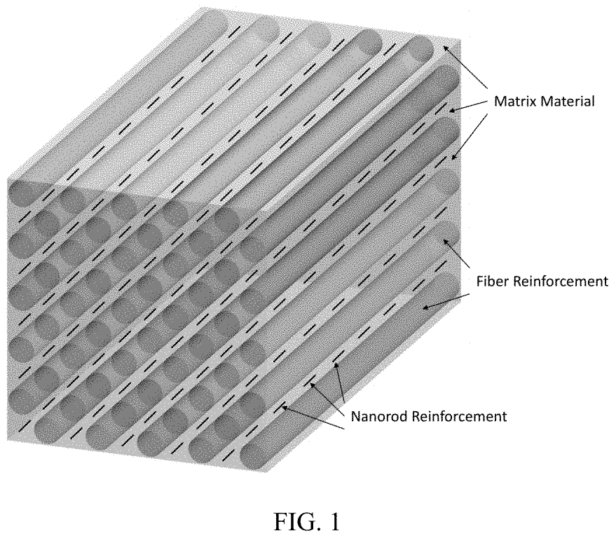

The above-described and other features will be appreciated and understood by those skilled in the art from the following detailed description, drawings, and appended claims.

### DETAILED DESCRIPTION

Described herein are bioresorbable composite materials having high strength and stiffness for bone fixation applications. Silk fibroin (SF) has been proven to be a degradable polymer with superb mechanical properties in tension. In the clinical setting and in the literature, silk has been shown to be excellent for use as sutures and tissue engineering scaffolds due to mechanical properties that are superior to most other bioresorbable polymers. However, SF has not been previously used to make a high-performance, dense composite in the field of biomaterials. With this in mind, the inventors used SF as the primary reinforcement material in composites made for load-bearing fixation applications. Methods for creating high-performance composites were developed with the aid of a design of experiments (DOE), with further work focusing on the use of HA particle reinforcement in tandem with SF fiber reinforcement.

In an aspect, a load-bearing bone fixation composite comprises a polymer matrix; a plurality of polymer fibers aligned along a common axis and disposed in the polymer matrix wherein the polymer matrix binds the surface of the polymer fibers; and a plurality of high aspect ratio nanorods coating at least a portion of each of the polymer fibers, wherein the long axis of at least a portion of the nanorods is partially or fully aligned with the common axis, and wherein the high aspect ratio nanorods have an aspect ratio of 10 or greater.

As used herein the polymer matrix provides the medium for disposing the polymer fibers and, as such, substantially contacts both the polymer fibers and the nanorods. The polymer matrix can also serve as the “glue” that holds the composite together. The matrix is thus selected for the ability to bind to the polymer fibers. In an aspect, the polymer matrix comprises polylactic acid (PLA), poly(ε-caprolactone) (PCL), aliphatic polyesters, polyethers, polycarbonates, their co-polymers other biodegradable polymers with a melting temperature less than 170° C., or a combination thereof. In the case of silk fibroin fibers, a preferred polymer matrix is PLA which binds well to silk fibroin fibers. PCL does not bind well to silk fibroin fibers.

Mixtures of matrix polymers can be employed depending on the viscoelastic properties of the system. In specific aspects, the matrix polymer can have a relatively low melting temperature (≤170° C.) and a relatively low viscosity to allow for good fiber and nanoparticle wetting. A higher Young's modulus such as greater than 1 GPa may be employed to provide suitable mechanical stability. However, when using a PLA matrix, silk fibroin fibers, and hydroxyapatite (HA) nanorods, the silk fibroin fibers and the HA nanorods provide sufficient mechanical stability, while the PLA “glues” the composite together.

In an aspect, the polymer matrix comprises about 5 to about 40% of the volume of the composite, specifically about 30%. Alternatively, the polymer matrix comprises about 10 to about 40 wt % of the total weight of the composite, specifically about 17 wt %.

The polymer fibers are continuous, biodegradable natural or synthetic polymer fibers such as poly(L-lactic) acid (PLLA) fibers, silk fibroin fibers, polyglycolic acid (PGA) fibers, polydioxanone (PDO) fibers, or a combination thereof. Optionally, the polymer fibers can further comprise metal wires such as magnesium wires.

In a specific aspect, the fibers comprise silk fibroin fibers from the cocoon of the Bombyx mori (B. mori) silkworm. The fibers have been degummed (the sericin coating has been removed). Degummed silk fibroin is not to be confused with regenerated silk fibroin or spider silk. Exemplary fiber diameters are 2 to 50 μm. In an aspect, the diameter of the fibers is 10.5±0.65 μm. In an aspect, in the composite, the spacing between the fibers is 1 to 10 μm depending on the processing. In the case of silk fibroin fibers, because they are sourced from cocoons and are not synthetic, the fibers are not perfectly straight and have a distribution throughout the composite. The density of silk fibroin is typically 1.4 g/cm³.

In an aspect, the polymer fibers comprise silk fibroin fibers and PLLA fibers.

In an aspect, the polymer fibers comprise about 30 to about 70% of the volume of the composite, specifically about 50%. Alternatively, the polymer fibers comprise about 30 to about 60 wt % of the total weight of the composite, specifically about 53 wt %.

The composite also includes a plurality of nanorods coating at least a portion of each of the polymer fibers, wherein the long axis of at least a portion of the nanorods is aligned with the common axis. Nanorods are nanoscale objects having a width of less than 1-100 nm. Nanorods can have all dimensions less than 1-100 nm. Nanorods have an aspect ratio (length divided by width) of greater than 5. High aspect ratio nanorods have an aspect ratio of 10 or greater, such as 30, 50, or even higher. Nanorods include nanowhiskers which are nano-scale in width, but not in length.

The inventors have found that high aspect ratio nanorods increase alignment within the composites and also increase the mechanical strength of the composites. Exemplary nanorods include bioglasses, calcium sulfates, calcium phosphates, calcium silicates, and the like. Exemplary calcium phosphates include hydroxyapatite, ion-substituted apatite, carbonate hydroxyapatite, fluorinated hydroxyapatite, chlorinated hydroxyapatite, silicon-containing hydroxyapatite, tricalcium phosphate, tetracalcium phosphate, monotite, dicalcium phosphate, dicalcium phosphate dihydrate, octacalcium phosphate, calcium phosphate monohydrate, alpha-tricalcium phosphate, beta-tricalcium phosphate, amorphous calcium phosphate, biphasic calcium phosphate, calcium deficient hydroxyapatite, precipitated hydroxyapatite, and oxyapatite, and combinations thereof. A preferred calcium phosphate is hydroxyapatite.

Hydroxyapatite (HA) provides naturally high mechanical properties and a slow degradation rate that matches the desired degradation rate for the application of bone fixation devices (it generally degrades in vivo in 1-2 years). For the composites, typically higher aspect ratio particles are better as they provide better stress transfer and better crack deflection and fiber pull-out. An exemplary aspect ratio for HA nanoparticles is 50 or higher.

In an aspect, the HA nanorods are HA nanowhiskers with the dimensions of 6.8 μm and 220 nm, leading to a final aspect ratio of 31.5.

In an aspect, the volume ratio of PLA matrix to HA nanorods can be around 3:2.

In an aspect, the nanorods comprise about 5 to about 30% of the volume of the composite, specifically about 20%. Alternatively, the nanorods comprise about 5 to about 40 wt % of the total weight of the composite, specifically about 30 wt %.

The composites can optionally comprise additional materials such as collagen, hyaluronans, fibrin, chitosan, alginate, silk, polyesters, polyethers, polycarbonates, polyamines, polyamides, co-polymers, poly(L-lactic) acid (PLLA), polyglycolic acid (PGA), poly(D,L-lactide-co-glycolide) (PLGA), and poly(ε-caprolactone) (PCL), and other polymers.

The composites described herein advantageously have a stiffness to make them suitable for medium-load-bearing applications. The stiffness of the material can be increased dramatically with just a slight increase in thickness because the stiffness is directly correlated to the thickness cubed (i.e., increasing the thickness of a material by just 50% will increase the stiffness by over 350%). Importantly, the materials described herein have a modulus, strength and toughness that are suitable for medium load-bearing applications.

The composites described herein are tunable with a modulus anywhere from 7 GPa to 21 GPa, depending on the composition of the composite that is used. This would enable the device to be tailored depending on if it were going to be used for maxillofacial fractures or load-bearing fractures. In an aspect, the composites have a flexural modulus of 7 GPa to 21 GPa, and/or a flexural strength of 200-550 MPA measured according to ASTM D7264.

In an aspect, the composite in a non-porous densified composite.

Also included herein are bone fixation devices comprising the composites described herein.

A method of bone fixation, comprising affixing the composite as described herein to a site of a load-bearing bone fracture, or maxillofacial bone fracture. Exemplary applications are in long-bone repair, spinal fusion, sternal bone closure, and maxillofacial fixation.

Also included herein are methods of making the composites.

A method of making a load-bearing bone fixation composite comprises coating a plurality of polymer fibers with a suspension containing a polymer matrix, a plurality of high aspect ratio nanorods, and a solvent for the polymer matrix, wherein the polymer matrix binds the surface of the polymer fibers, and wherein the high aspect ratio nanorods have an aspect ratio of 10 or greater,

optionally repeating the coating,

aligning the plurality of coated polymer fibers, and

consolidating the plurality of coated polymer fibers to provide the composite.

In an aspect, aligning the plurality of coated polymer fibers comprises wrapping the plurality of coated polymer fibers on a frame, e.g., a metal frame. Consolidating can comprise placing the frame in a mold and pressing the fibers under heat and pressure, wherein the press temperature melts the polymer matrix but not the polymer fibers.

The invention is further illustrated by the following non-limiting examples.

### EXAMPLES

### Methods

Materials:

The following materials were purchased from Fisher Scientific: calcium nitrate tetrahydrate (Ca(NO₃).4H₂O, ACS certified), ammonium phosphate dibasic ((NH₄)₂HPO₄, ACS certified), methyl ethyl ketone (MEK, ACS certified), and ammonium hydroxide (NH₄OH, certified ACS plus). Cetyltrimethylammonium bromide (CTAB, ≥99%) was purchased from Sigma Aldrich. Dichloromethane (DCM, stabilized with amylene, ≥99.8%, for analysis) was purchased from Arcos Organics. Both poly-L-lactic acid (PLLA) and silk fibroin (SF) fibers were generously supplied by Teleflex Medical Inc, and polycaprolactone (PCL, M_{w}=80,000) was purchased from Instamorph®.

HA Synthesis and Surface Treatment:

Hydroxyapatite nanorods were synthesized via a wet precipitation method as previously described in the art. Briefly, 188.86 g of Ca(NO₃)₂.4H₂O was dissolved in 720 mL deionized water (DiW), where 24 mL of NH₄OH was added. Next, 696 mL DiW was added to dissolve the resulting precipitate, completing the formation of solution 1. To make solution 2, 63.36 g of (NH₄)₂HPO₄ was dissolved in 1200 mL of DiW. 600 mL of NH₄OH was added to the solution, and subsequently 760 mL of DiW was added to dissolve the resulting precipitate, concluding solution 2. Next, solution 2 was added drop-wise to solution 1, and, upon completion, boiled for 3 hours. Once cooled, the HA nanorods formed were surface treated to ensure even distribution and minimal aggregation in the dip-coating suspension, as previously described in the art. Briefly, the supernatant of the resulting suspension of HA particles from the procedure previously outlined was poured off until a final HA concentration of ˜3 g/dL was reached (final volume of ˜2.5 L). Next, 4 g of CTAB was suspended in 60 mL of DCM and quickly added to the HA suspension under vigorous stirring. The suspension continued stirring for 2 h, and then was left to sit overnight at 60° C. to aid in the surface treatment. Following the surface treatment, the HA was washed with DiW 5 times to remove the NH₄OH and dried at 150° C. overnight. Based on these previous studies, the final particles take the shape of nanorods with a final length, width, and aspect ratio of 70 nm, 12 nm, and 6, respectively.

Design of Experiments:

Many materials were considered for the development of high-performance bioresorbable composites, with the final list of materials to be specifically tested being SF fibers, PLLA fibers, PLA matrix, PCL matrix, and HA nanoparticles. These materials were selected because they have been shown in literature to safely degrade in vivo and are all already FDA-approved materials. Due to the many different materials used and possible formulations, a design of experiments (DOE) was utilized to assess the effect of key variables in the processing of the composite on the 3 mechanical properties (i.e. the dependent variables) of interest: flexural modulus, flexural strength, and relative flexural toughness. In total, five independent variables were chosen, including fiber type ratio (SF:PLLA vol % ratio), matrix type, matrix amount, and HA amount, as well as a blocking variable, which was the mold cavity within the compression mold.

For each of these variables, a “low” and a “high” value was chosen to use in the DOE. The SF:PLLA ratio was either 1:9 or 9:1, as these ratios allowed for the minimum of one full layer for each fiber type in the final composite bar (i.e., one layer of SF fibers for the 1:9 ratio and one layer of PLLA fibers for the 9:1 ratio). Matrix type, a categorical variable, was either PCL or PLA, test how matrix type affected the bending properties of the composite. Preliminary testing showed less than 2 wt/vol % matrix (PCL or PLA) in the dip coating suspension did not wet fibers sufficiently, and more than 10 wt/vol % matrix in the dip-coating suspension caused complications in processing the composite. So, these values were chosen as the limits in the DOE. Similarly, preliminary tests showed that having greater than 15 wt/vol % HA in the dip-coating suspension caused complications in processing, as well as severe aggregation of HA in the final composite. As such, the HA amount for the DOE was either 0 wt/vol % or 15 wt/vol % in the dip-coating suspension. Lastly, the mold cavities were simply labeled A or B and were used to confirm that the mold cavity would not affect the properties of the composite samples (i.e. the samples should have the same properties regardless of which mold cavity they are pressed in).

To minimize the number of runs required to complete the DOE, an assumption was made that there would be negligible-to-zero three-way interactions of the factors on the three dependent variables of interest. As such, the number of runs was reduced from 2ⁿ to 2ⁿ⁻¹, where n is the number of independent variables, for a total of 16 runs for the DOE. The nm list and sample compositions are listed in Table 1, however, the runs were completed in a randomized order to control for error. DOE analysis was performed using DOE analytics tools in the statistical analysis software Statistica™. Factors were said to have a significant effect on the dependent variables if the p-value was ≤0.05. Factors were said to still affect the dependent variable if the p-value was between 0.05 and 0.20, but no definitive conclusions can be directly drawn since these p-values were relatively high (i.e. above 0.05). As such, factors with these p-values warrant further investigation with refined variables and experimental design. Initial analysis of effects showed three factors (one factor for each dependent variable) had p-values that were nearly 1. As such, and as is common when performing DOE analysis, if a factor was deemed to have negligible effects on the dependent variable (i.e. it had a p-value close to 1), it was rolled into error, as indicated by the dashes in Table 2. By rolling these factors into error, the analyses for the remaining effects are more discriminating (i.e. the DOE analysis will provide more accurate results for the remaining factors).

TABLE 1
A list of the compositions that were made and
tested for DOE in this study. Note, the order of the
samples was randomized to further reduce outside factors.

| | | | | Matrix | HA Amount |
|---|---|---|---|---|---|
| | | | Matrix | Amount in | in |
| Run | Mold | SF:PLLA | Material | Suspension | Suspension |
| Number | Cavity | Ratio | Used on SF | (wt/vol %) | (wt/vol %) |
| 1 | 1 | 9:1 | PCL | 2 | 15 |
| 2 | 2 | 9:1 | PCL | 2 | 0 |
| 3 | 1 | 1:9 | PCL | 2 | 0 |
| 4 | 2 | 1:9 | PCL | 2 | 15 |
| 5 | 1 | 9:1 | PLA | 2 | 0 |
| 6 | 2 | 9:1 | PLA | 2 | 15 |
| 7 | 1 | 1:9 | PLA | 2 | 15 |
| 8 | 2 | 1:9 | PLA | 2 | 0 |
| 9 | 1 | 9:1 | PCL | 10 | 0 |
| 10 | 2 | 9:1 | PCL | 10 | 15 |
| 11 | 1 | 1:9 | PCL | 10 | 15 |
| 12 | 2 | 1:9 | PCL | 10 | 0 |
| 13 | 1 | 9:1 | PLA | 10 | 15 |
| 14 | 2 | 9:1 | PLA | 10 | 0 |
| 15 | 1 | 1:9 | PLA | 10 | 0 |
| 16 | 2 | 1:9 | PLA | 10 | 15 |

TABLE 2
The factors that have an effect on the dependent variables of
interest, where the bolded numbers represent factors that
influenced the dependent variable and blank spaces indicate
that particular factor had virtually zero effect on the results.

| | Modulous | Strength | Toughness |
|---|---|---|---|
| Factor | p Value | p Value | p Value |
| Mold Cavity | 0.68820 | 0.74119 | 0.21902 |
| 1. PLLA:SF Ratio | 0.00046 | 0.00433 | 0.00005 |
| 2. Matrix Type | 0.17060 | 0.15695 | 0.06096 |
| 3. Matrix Amount | 0.49650 | 0.19079 | 0.00454 |
| 4. HA Amount | 0.05950 | 0.10756 | 0.21920 |
| 1 by 2 | — | 0.26714 | 0.45178 |
| 1 by 3 | 0.39206 | — | 0.30762 |
| 1 by 4 | 0.20501 | 0.50118 | 0.12081 |
| 2 by 3 | 0.11765 | 0.52166 | — |
| 2 by 4 | 0.45405 | 0.46637 | 0.01613 |
| 3 by 4 | 0.23746 | 0.11100 | 0.00514 |

Composite Processing and Fabrication:

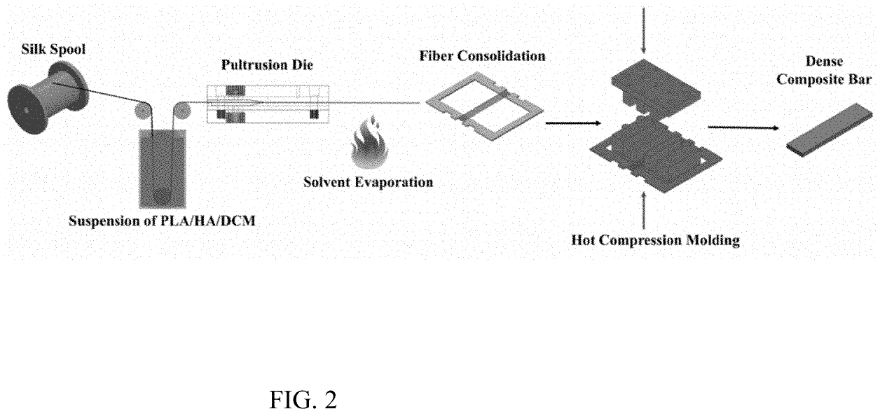

The general procedure for the composite processing is shown in FIG. 2, but it is important to note that this figure does not explicitly contain every step of the composite processing as it will be explained in greater detail in writing. To start, PLLA fibers were run through a dip-coating solution of 2 wt/vol % PCL in MEK, as 2 wt/vol % was shown to have sufficient fiber wetting in the art. The PLLA fibers were then run through the pultrusion die, dried, and this process was repeated a second time before the fibers were consolidated on a steel frame for the compression molding step. Next, SF fibers were coated in a similar fashion with a dip-coating suspension containing either PCL or PLA in DCM with and without HA (using the concentrations previously described) depending on the run number as shown in Table 1. After the second coating, the SF fibers were wrapped around the previously coated PLLA fibers on the metal frame, with the SF:PLLA fiber ratio being determined by the number of wraps of the respective fibers around the metal frame. As shown in FIG. 2, the steel frame contained slots for the consolidated fibers that were the same width as the slots in the compression mold. As such, once the fibers were consolidated on the steel frame, the frame was placed around the mold so the fibers lined up with the mold slots. When the fibers were properly placed in the slots, the consolidated fibers were then pressed at 160° C. and 200 MPa. The pressing temperature was chosen based on the melting temperatures of the thermoplastics used in this experiment. PCL, PLA, and PLLA have melting temperatures of ˜60° C., 150-160° C., and ˜170° C., respectively. As such, using a pressing temperature of 160° C. ensures the matrix polymers (PCL and PLA) will melt and wet the reinforcement fibers, while the PLLA fibers remain largely unharmed. The resulting composite bars were then tested via three-point bending to determine the flexural mechanical properties.

HA Content Optimization:

Due to the HA parameters set for the DOE, the results for the effect of HA on the properties of the composite could not be determined with the DOE. As will be discussed in greater detail in the results section, the addition of HA should have caused the modulus and strength to increase in such a composite as a ceramic reinforcement phase has a significantly higher modulus and strength compared to the polymeric matrix. However, results from the DOE showed the contrary; HA had either negligible or detrimental effects on the mechanical properties of the composites. As such, a second study was carried out in order to optimize the HA content within the composite. The processing remained largely the same as previously described for the DOE, however, PLLA fibers were omitted, focusing solely on monolithic SF/PLA/HA composites based on the DOE results. Briefly, SF fibers were dip-coated in a suspension containing five different concentrations of HA suspended in a solution of PLA dissolved in DCM, including 3, 6, 9, 12, and 15 wt/vol %. The five different amounts of HA were also compared to a control composite containing no HA. It is worth noting that the PLA content in the dip-coating suspension for this portion of the study remained constant at 6 wt/vol %, picking a concentration directly in the middle of the “low” and “high” values from the previous DOE. Following the fabrication, the composites underwent flexural testing using the same procedures outlined previously, and the flexural modulus and strength for each composition were plotted against the HA vol % for each composition. Also, the flexural toughness was determined using the same method as previously described for the DOE, and the results were also plotted against HA content.

Characterization:

Flexural tests were performed on an Instron® 1011 with a three-point bending fixture. The tests were performed with a support span of 45 mm and at a loading rate of 20 mm/min, which were determined based on ASTM standard D7264. From the load and displacement data obtained, flexural stress versus strain data were plotted where the flexural modulus was taken as the linear region of this plot. The flexural strength was taken as the maximum stress value obtained during the test, and the toughness was calculated as the area under the stress versus strain curve up to 80% of the maximum stress after the sample yielded.

Thermogravimetric analysis (TGA) was carried out on a TA instruments Q500 machine to determine the HA wt % for different compositions prepared for this study. To ensure a homogenous distribution of HA within the samples, two pieces from two samples of each composition were tested with TGA for a total sample size n of 4. The procedure for TGA involves heating each sample to 800° C. at a rate of 15° C./min, causing the PLA and SF to burn off and determining the wt % of HA within each sample. The wt % provided by the TGA were then converted to vol % using the following density values for HA, SF, and PLA, respectively: 3.156 g/cm³, 1.40 g/cm³, and 1.290 g/cm³. Since each composition has the same amount of SF and the amount of HA for each composition is determined through TGA, the volume percent of each component was calculated using the previously-mentioned densities for each component in tandem with the physical dimensions of the composite bars.

Field emission scanning electron microscopy (FESEM) was performed on the individual SF and PLLA fibrils as well as on composite samples using an FEI Teneo LVSEM. The average diameter of each fibril type was determined by size analysis on 50 fibrils for each type using ImageJ. Samples for transverse cross-sectional images were prepared by lowering a razor blade mounted in a milling machine unto the sample to obtain a clean cross-section. Samples for longitudinal cross-sectional images were prepared by mounting composite samples in epoxy and polishing the surface using sandpaper and eventually polishing with a suspension containing 5 μm sized gold particles. Since the samples are nonconductive they were coated with gold-palladium (SEM coating unit E5100, Polaron Instruments Inc.) prior to imaging, and were imaged at an accelerating voltage of 5 kV. The hydroxyapatite nanoparticles produced for this study were imaged using the FEI Tecnai T12 scanning transmission electron microscope (STEM). Fourier transform infrared spectroscopy (FTIR) was utilized to prove the efficacy of the surface treatment of HA with CTAB.

Statistical Analysis:

Results from the three-point bending tests were statistically analyzed using two-way analysis of variance (ANOVA) and expressed as mean±standard deviation. Statistical significance was defined as p<0.05.

### Results

### Example 1: Materials Characterization

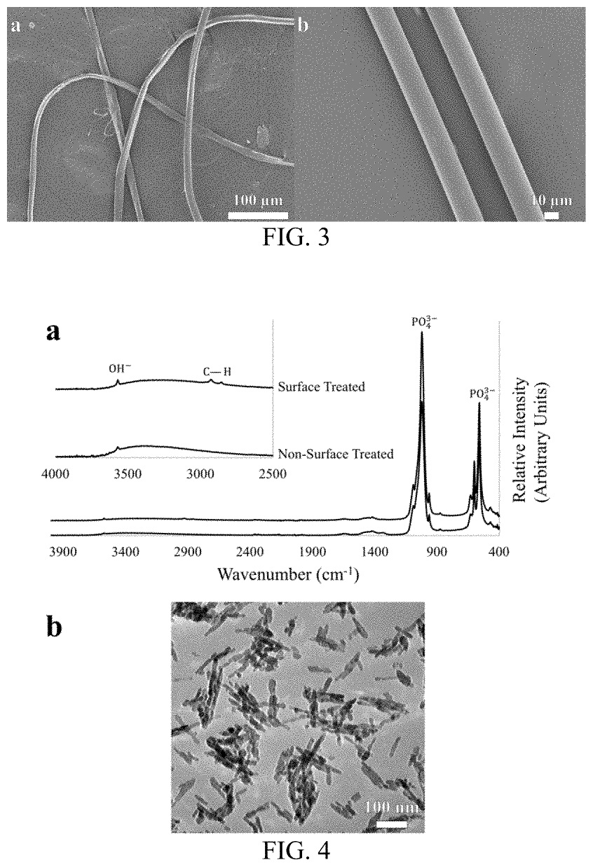

FIG. 3 shows FESEM images of SF and PLLA fibrils. From these images, it is apparent that SF has a slightly irregular shape as it is a natural fiber, and it has an average diameter of 10.5=0.65 μm. Conversely, the PLLA fibers display a perfectly cylindrical shape with an average diameter of 17±0.69 μm. FIG. 4 shows the TEM images and FTIR spectroscopy performed on the HA nanoparticles. The TEM image shows the rod-shaped nanoparticles with an aspect ratio of ˜6. The FTIR spectra confirm that HA was produced and successfully surface treated with CTAB based on the presence of peaks at 2850 cm⁻¹ and 2950 cm⁻¹ corresponding to C—H bonds that are present in CTAB.

### Example 2: Design of Experiments

As previously mentioned, the DOE employed in this study was utilized to observe the effects of multiple factors in the process of making the composite materials outlined above at the same time, including two-way interactions between the factors. Table 2 shows the p values of effects different factors and two-way interactions had on the three dependent variables, where bolded numbers represent factors that had an effect on the respective dependent variable. Also, the values shown in Table 2 represent the p values after negligible factors were removed from the model, which are indicated by the blank spaces in the table. After ignoring the negligible factors for the respective dependent variables, the p values for the remaining factors further decreased, indicating a slightly more accurate DOE model for this particular system.

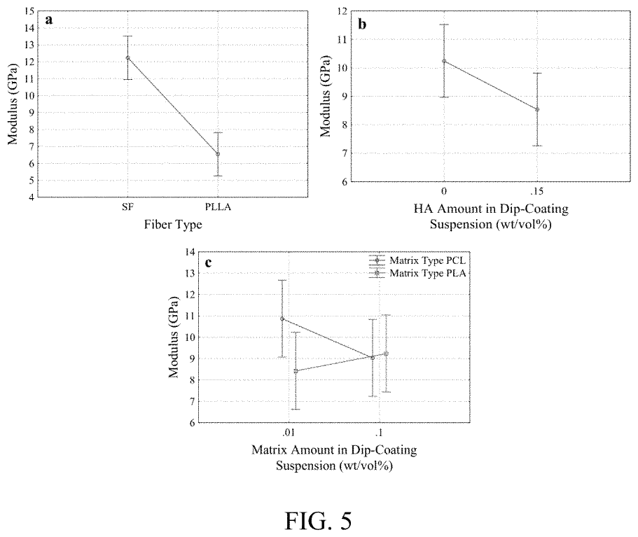

FIG. 5 shows factors that have a significant effect on the flexural modulus of the composite material. From FIGS. 5a and 5b, respectively, increased SF fiber content caused an increase in flexural modulus relative to PLLA fibers and the flexural modulus slightly decreased when HA was added to the system. FIG. 5c shows the two-way interaction between matrix type and matrix amount, where the flexural modulus decreased when adding more PCL to the system, but increased when adding more PLA to the system.

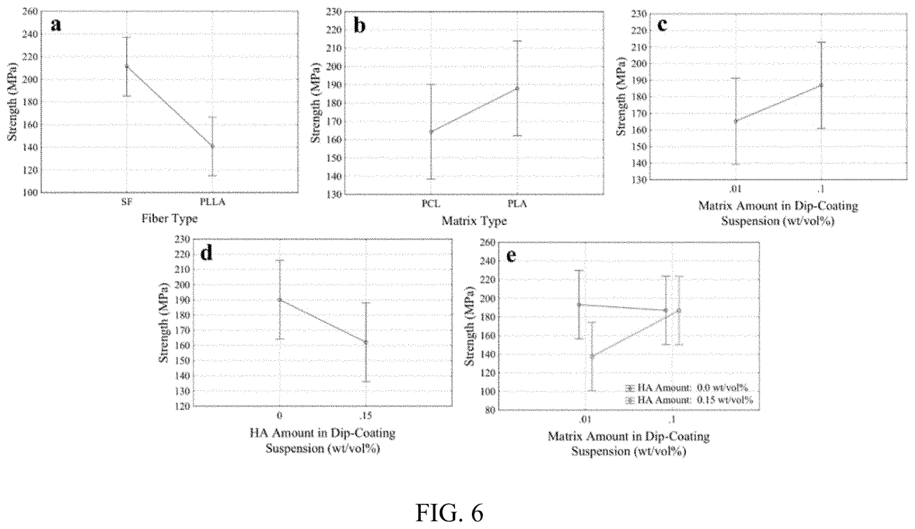

FIGS. 6a-e show the effects four of the factors and one two-way interaction had on the flexural strength of the composite. FIGS. 6a, 6b, 6c, and 6d, respectively, show that the use of more SF fibers over PLLA fibers increases the strength, the use of PLA as the matrix polymer increases the strength versus when PCL is used, increasing matrix content increases the strength, and the addition of HA causes the flexural strength to decrease. However, FIG. 6e displays the effect of the two-way interaction between matrix amount and HA amount, showing a negligible effect when HA is absent from the system and matrix content is increased, but showing a significant increase in strength when HA is present and matrix content is increased.

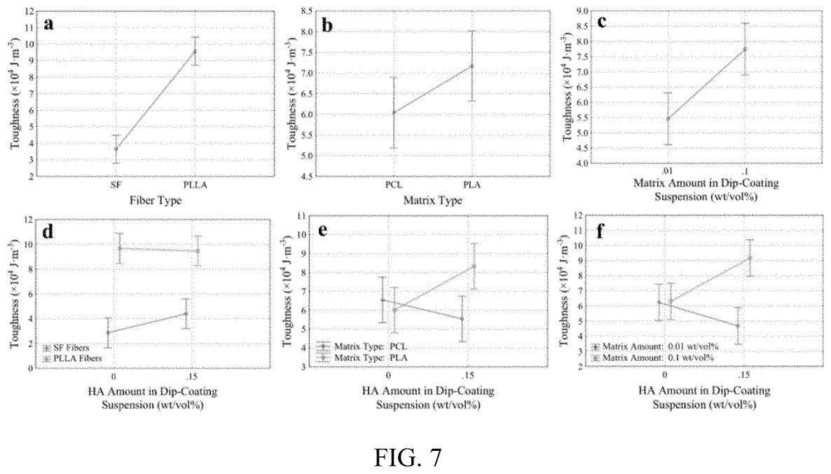

FIGS. 7a-c show the effects three factors, namely fiber type, matrix type and matrix amount, had on the toughness of the composites, while FIGS. 7d-f show the effect two-way interactions had on the toughness of the composite. Contrary to the effects on flexural modulus and strength, the use of PLLA fibers was shown to cause superior toughness over the use of SF fibers. Also, using PLA as the matrix increased the toughness over using PCL, and increasing the matrix amount increased the toughness. FIG. 7d shows that the addition of HA increased the toughness when SF fibers were used as the reinforcement, but had little effect when PLLA was the predominant reinforcement. FIG. 7e shows that the addition of HA caused a decrease in toughness when PCL was used as the matrix polymer, however, adding HA to the system increased the toughness when PLA was used as the matrix polymer. FIG. 7f shows that the addition of HA increased toughness of the composite when more matrix polymer is present, but HA was detrimental for toughness when there was a small amount of matrix polymer present.

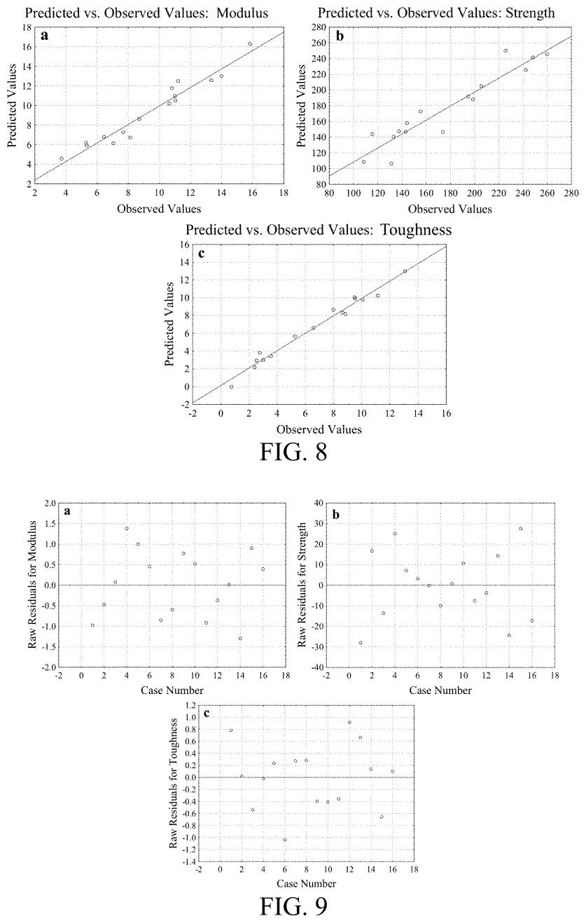

FIG. 8 a-c show how well the experimental results match up with the DOE model, giving a predicted values versus observed values chart for each modulus, strength, and toughness investigated in this study. Also, FIGS. 9a-c shows the residual values (error) versus case number for modulus, strength, and toughness, demonstrating that the error was consistent throughout the entire study, leading us to believe there were negligible outside influences on the outcome of the study and the factors analyzed in this DOE were the main contributors to the values of the dependent variables.

### Example 3: HA Optimization

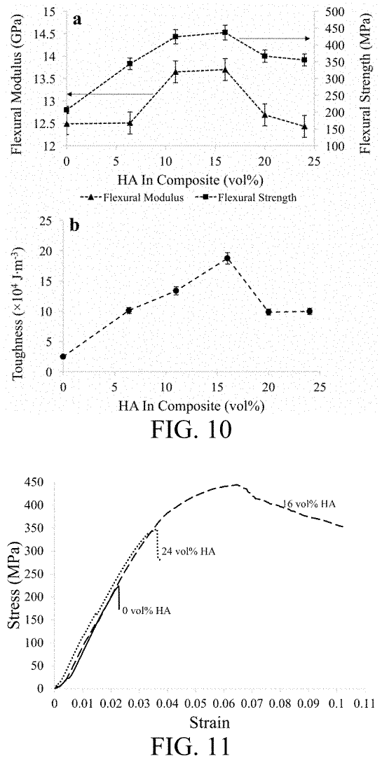

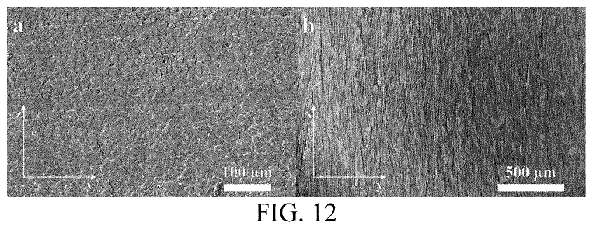

FIG. 10a shows the effect of HA content on flexural modulus and strength. As seen in the figure, the addition of HA exhibits an immediate impact on flexural strength, showing a statistically significant increase when HA is added to the system. However, the addition of HA initially shows no significant effect on flexural modulus. For both flexural strength and modulus, the peak values were 437.0 MPa and 13.7 GPa, respectively, with no significant difference between samples with 11 vol % HA versus 16 vol %. The modulus and strength were both shown to decrease when the HA content exceeded 16 vol % in the sample. Similarly, the addition of HA to the composite samples caused a significant increase in the flexural toughness of the composite material, as seen in FIG. 10b. The maximum toughness occurs when 16 vol % HA is used in the dip-coating suspension with a value of 18.7×10⁴ J·m⁻³, with a sharp decrease occurring when the HA exceeds this value. The summary of the results from this study is seen in Table 3, where the optimal composition, based on flexural modulus, strength, and toughness, is indicated by the bolded row. Furthermore, FIG. 11 shows sample stress vs. strain curves for the control sample (0 vol % HA), the optimal sample (16 vol % HA), and sample with the highest amount of HA in the composite (24 vol %) up to 80% strength retention after failure. As shown in the figure, the toughness and strength of the composites are significantly higher when 16 vol % HA is present versus when no HA and excess HA is present in the composite. Also, FIG. 11 shows the delamination mode of failure most samples suffered from. Lastly, the longitudinal and transverse structure of a composite with this optimal formulation is shown in FIGS. 12a and 12b, respectively. From the figures, it is apparent that the SF fibers are largely oriented in the x direction, and the reinforcement is distributed evenly throughout.

TABLE 3
Summary of the results from the 3-point bending tests, showing the
effect of HA vol % within the composite on flexural modulus, strength,
and toughness. The optimal composition in this study contained
16 vol % HA, indicated in the table by the bolded numbers.

| SF | HA | PLA | | | |
|---|---|---|---|---|---|
| Content | Content | Content | Modulous | Strength | Toughness |
| (vol %) | (vol %) | (vol %) | (GPa) | (MPa) | (× 10⁴ J · m⁻³) |
| 68 | 0 | 32 | 12.49 | 206.7 | 2.48 |
| 58 | 6 | 36 | 12.51 | 344.1 | 10.11 |
| 55 | 11 | 34 | 13.65 | 424.2 | 13.36 |
| 51 | 16 | 33 | 13.70 | 437.0 | 18.74 |
| 49 | 20 | 31 | 12.69 | 366.7 | 9.85 |
| 48 | 24 | 28 | 12.43 | 355.3 | 9.96 |

### Discussion of Example 1-3

The reported elastic modulus for degummed SF in the literature is significantly higher than that of the PLLA fibers used in this study, with respective moduli of 16-22.6 GPa and 8.1 GPa for SF and PLLA. As such, the DOE showed that samples made with predominantly SF fibers, rather than PLLA fibers, had significantly higher flexural moduli compared to samples made with predominantly PLLA fibers, as expected. However, the addition of HA had the unexpected consequence of slightly decreasing the flexural modulus, which may be due to the nature of this particular DOE. The amount of HA used in the DOE was chosen to be 15 wt/vol % in the dip-coating suspension, and high concentrations of nanoparticles cause the HA to agglomerate and decrease the mechanical properties of composite materials. This unexpected result of HA decreasing the modulus was the driving force for performing the latter study relating flexural properties of SF/HA-reinforced composites to HA content. Another unexpected result of the DOE was the inconsistent effects PLA and PCL had on the modulus of the composite. According to the rule of mixtures, and assuming proper fiber wetting, it is expected that increasing the matrix content in the composite will decrease the modulus as the matrix has a relatively lower modulus compared to that of the fibers. However, as previously mentioned, while increasing PCL provided this expected result, increasing the PLA content caused a slight increase in modulus. Without being held to theory, this inconsistency can be explained by the differences in viscosity of the two matrix polymers, where the PCL has a much higher viscosity than the PLA. At the “low” amounts of matrix for the DOE, it is possible there was not enough matrix to properly wet the fibers, especially when HA was present in the system. As such, when PLA content was increased (i.e. to the “high” value of the DOE), the matrix could more sufficiently wet the fibers, while the pressing step will expel a lot of the excess matrix, thus increasing the modulus over the “low” amount. However, due to the drastic increase in viscosity over PLA, when the PCL content is increased, excess matrix material is less likely to be removed from the system, thus causing a decrease in modulus, as expected based on the rule of mixtures.

Samples prepared with SF as the long fiber reinforcement had higher flexural strength values than those prepared with PLLA. SF is shown in literature as having a much higher tensile strength than PLLA. Similarly, the use of PLA as the matrix material proved to produce samples with higher strength values over those made with PCL due to the high strength of PLA relative to PCL (i.e., PLA and PCL have reported tensile strengths of 32.5 MPa and 10.5 MPa, respectively). The DOE also revealed that increasing the matrix content improved the strength of the composite, which is due to increased fiber wetting and a subsequent increase in interfacial shear strength within the composite. Similar to the effect HA had on the flexural modulus in this DOE, the addition of HA had a detrimental effect on the flexural strength due to the formation of aggregated HA particles, which has been shown to be severely damaging to mechanical properties of composites. FIG. 10e shows the two-way interaction between HA amount and matrix amount. This figure shows that when there is a small amount of matrix polymer present the addition of HA is significantly detrimental to the strength of the composite. However, when HA is present with a high amount of matrix polymer the strength increases to a statistically equal value relative to when HA is not present in the composite, which is likely due to superior wetting of the fibers and particles when more matrix is present with HA.

Without being held to theory, it is believed that the toughness of the composites suffered greatly when SF fibers were the predominant fibers in the sample because of the tendency for the samples to undergo Mode II delamination, a phenomenon not seen in samples made with PLLA. The apparent increase in toughness when using PLA as the matrix is believed to be a result of the increased strength. Due to the delamination of the samples with SF reinforcement, there was little plastic deformation. As such, the increased strength when using a PLA matrix resulted in an increased toughness as the PLA simply postponed the mode II delamination of the sample compared to samples with a PCL matrix. As seen previously with the effect of matrix amount on strength, increased matrix content resulted in tougher composite materials, likely due to increased wetting of the fibers and particles. FIG. 8d displays the two-way interaction between long fiber reinforcement type and HA amount, showing the addition of HA when SF is the primary reinforcement material increases toughness while it has a negligible effect when PLLA is the predominant reinforcement material due to the inherently great toughness of the PLLA fibers used. The presence of HA particles enables crack deflection to occur within the composite, which is shown in literature to increase the toughness within composites. The effect of HA amount and matrix type on toughness is another two-way interaction present in the study. The DOE showed that the presence of HA when using PLA as the matrix increased the toughness of the composite, but when using PCL as the matrix material the toughness showed a slight decrease. Without being held to theory, it is believed that this is caused by the drastic increase in matrix viscosity when particle reinforcement is present in a polymer matrix, which is known to hinder fiber wetting within composites, as well as the previously-mentioned agglomeration issue present with such a high HA content. Due to the significantly higher viscosity of PCL, the addition of HA makes it even more difficult to properly wet the fibers and particles. FIG. 8f shows the same relationship as FIG. 10e, where the addition of HA increases toughness when there is a high amount of matrix present, but the addition of HA is detrimental when there is a low amount of matrix. This is due to the aforementioned fiber/particle wetting issue when using low amounts of matrix, which was the driving force for the need to perform a study to optimize the HA content within the composite.

FIG. 12a shows the relationship between the flexural modulus and strength and HA content. From the figure, when 6 vol % HA is present in the composite, there is a negligible effect on the modulus relative to samples with no HA. However, when the composites contained 11 vol % and 16 vol % HA the flexural modulus increased to 13.65 GPa and 13.7 GPa, respectively, both of which are higher than values found in literature for bioresorbable bone fixation composites. When the HA exceeds these values, the flexural modulus decreases significantly due to a combined effect of HA agglomeration and poor particle wetting by the matrix polymer. When examining flexural strength, the strength significantly increases from 207 MPa to 344 MPa when HA content increased from 0 to 6 vol %. The strength further increases to 424 MPa and 437 MPa when 11 vol % and 16 vol % are present, respectively. Upon further increasing the HA content to 20 and 24 vol %, the strength of the composites decreases to 367 MPa and 355 MPa, respectively. Following a similar trend, HA is shown to improve the toughness of the composite samples during bending, as seen in FIG. 12b. Without being held to theory, it is believed that the addition of HA immediately improves the toughness of the composite due to greatly improved crack deflection within the matrix, reaching a peak value of 18.7×10⁴ J·m³ when there is 16 vol % HA in the composite. It was noted in the prior art that while particle reinforcement significantly improves toughness due to crack deflection, particle agglomerates act more as stress concentrations than crack deflectors, which is the reason for the significant decrease in toughness beyond an HA content of 16 vol %. It is also important to note that while composites with 16 vol % HA were optimal for the present study, the mechanical properties could be improved further by adjusting the long fiber and matrix content as well.

Another important note is that while appropriate moduli and strengths were achieved, all of the samples except for the samples of optimal formulation failed due to mode II delamination, as discussed previously. While this remained true for many of the samples with 16 vol % HA in the composite as well, some plastic deformation was seen in these samples prior to delamination. This is shown in FIG. 11, where the 0 vol % HA and 24 vol % HA stress vs. strain curves show delamination before any notable plastic deformation, whereas the sample with the optimal formulation showed plastic deformation, which is the reason or such a large increase in toughness. Furthermore, the failure of the 16 vol % HA sample more closely resembles that of natural bone.

Improvements can be made to this composite by targeting the shortcomings pointed out in this study. The HA used in the present study is polycrystalline HA with an aspect ratio of 6. However, if HA particles were prepared to be single crystal and have a higher aspect ratio, the particles would have a significantly higher modulus and be more effective at transferring the load within the composite. Further improvements could be achieved by reducing HA aggregation within the composite. This could be done by increasing the matrix amount in the dip-coating suspension (i.e. increasing the viscosity of the suspension), which has been shown to decrease particle agglomeration and would preserve the matrix-to-reinforcement ratio required to make effective composites at high particle loadings.

TABLE 4
Summarizes the mechanical properties and particle orientation of various formulations
of silk/HA/PLA composites. Note, there are two different types of HA presented here.
The polycrystalline HA particles are ellipsoidal nanorods with an aspect ratio of 6,
and the single crystalline HA particles are nanowhiskers with an aspect ratio of 31.

| | | | | | | | Herman's | |
|---|---|---|---|---|---|---|---|---|
| | Silk | HA | PLA | | | | Orientation | |
| | Content | Content | Content | Modulus | Strength | Toughness | Factor of | |
| Samples | (vol %) | (vol %) | (vol %) | (GPa) | (MPa) | (kJ · m⁻²) | HA | Failure Mode |
| PLA/Silk | 88 | N/A | 12 | 16.5 | 223 | 6.0 | N/A | Delamination |
| Polycrystalline | 58 | 6 | 37 | 12.5 | 344 | 16.7 | 0.07 | Delamination |
| HA/PLA/Silk | | | | | | | | |
| Polycrystalline | 50 | 16 | 35 | 13.8 | 436 | 35.2 | 0.05 | Fibrous and |
| HA/PLA/Silk | | | | | | | | Delamination |
| Single Crystal | 54 | 12 | 34 | 18.4 | 450 | 26 | 0.36 | Fibrous |
| HA/PLA/Silk | | | | | | | | |
| Single Crystal | 51 | 16 | 33 | 19.6 | 461 | 25 | 0.38 | Fibrous |
| HA/PLA/Silk | | | | | | | | |
| Single Crystal | 48 | 20 | 30 | 20.8 | 518 | 29 | 0.42 | Fibrous |
| HA/PLA/Silk | | | | | | | | |
| Cortical bone | N/A | N/A | N/A | 7-25 | 90-283 | 11 | | Largely |
| | | | | | | | | Brittle |
| Targeted | | | | 25 | 250 | 11 | | Fibrous |
| composite | | | | | | | | |

### Example 4: Analysis of Additional Compositions

Starting at the top row of Table 4 with the SF/PLA composite with no HA present, the modulus of 16.5 GPa is expected based on the moduli of SF and PLA (18 GPa, and 1.4 GPa, respectively) and the rule of mixtures that can be applied to estimate the modulus. However, the flexural strength is rather low at 223 MPa due to the occurrence of delamination as the mode of failure. Adding polycrystalline HA nanorods to the composite increases the strength of the composite. The strength of the composite increases immediately with the addition of HA nanorods, reaching its maximum value of 436 MPa with the presence of 16 vol % HA. Without being held to theory, it is believed that this dramatic increase in bending strength is the result of the nanorods inducing crack deflection within the composite. Increasing the HA content beyond 16 vol % causes a decrease in strength due to HA agglomeration and poor particle wetting within the composite. While the strength increases with the addition of HA, the modulus appears to decrease when the HA nanorods are present. Without being held to theory, it is believed that this decrease in modulus is due to the relative silk fibroin fiber content decreasing and the poor reinforcement efficiency of the nanorods because of the comparatively low aspect ratio. Furthermore, the modulus of the HA nanorods is assumed to be much lower than the theoretical maximum of HA (˜40 GPa, instead of 140 GPa) because the particles are polycrystalline and not single crystalline.

Replacing the polycrystalline HA nanorods with single crystal HA nanowhiskers causes a significant increase in both bending modulus and strength. Due to the single crystal nature of the nanowhiskers, the HA comes much closer to its theoretical maximum value of 140 GPa. This significant increase in particle modulus, in tandem with the increased reinforcement efficiency caused by the higher aspect ratio of HA, increases the bending modulus of the composites to 20.8 GPa. The increase in aspect ratio from 6 to 31 causes the reinforcement efficiency to increase from 18 to 68.2, which also accounts for the increase in bending strength to 518 MPa. Another benefit of this increased aspect ratio is greater particle alignment within the composite, shown by the Herman's orientation in Table 4. The change in HA particle morphology increased the Herman's orientation constant from 0.07 to 0.42, where a value of 0 would indicate random orientation and a value of 1 would indicate perfect alignment. From this, it can be assumed HA nanorods have negligible alignment within the composites, while the HA nanowhiskers have partial alignment. The bending modulus of 20.8 GPa nearly matches that of natural bone, and the strength of 518 MPa greatly exceeds that of natural bone. Perhaps the most important benefit of using the HA nanowhiskers, versus the previously used nanorods, is the change in failure mechanism. The use of nanowhiskers causes the failure mechanism to change from delamination to fibrous failure, which eliminates the occurrence of catastrophic failure.

### Conclusions from Examples 1-4

This study showed the development of a load-bearing composite that is bioresobable using silk fibroin as the primary reinforcement material. A DOE was performed to determine the effects various factors had on the flexural modulus, strength, and toughness of the composites. In particular, this DOE analyzed how different long fiber reinforcement materials, matrix types, matrix amount, and HA amounts affected the three mechanical properties of interest. From the data obtained performing the DOE, a subsequent study was performed to optimize the HA particle reinforcement content for composites containing SF fibers and PLA matrix. Using SF, PLA, and HA, which are all currently FDA-approved materials, high-performance composites were fabricated that achieved a flexural modulus, strength, and toughness of 13.7 GPa, 437 MPa, and 18.7×10⁴ J·m⁻³, respectively, when using nanorods, or 20.8 GPa, 518 MPa, and 29 kJ·m⁻³, respectively, when using nanowhiskers. When nanowhiskers are used as the particle reinforcement phase, the modulus is in the range of the highest reported values for a bioresorbable composite for bone fixation and the strength far exceeds the requirements for such a device. FESEM images show the reinforcement fibers are aligned in the x direction, allowing the high stiffness and strength to be achieved. Such a composite material shows promise for use as a load-bearing material for fixation devices, providing a better long-term solution over the metal alternatives currently in place.

### Example 5: HA Synthesis and Characterization

Hydroxyapatite reinforcement nanowhiskers were prepared based on a modified protocol involving urea decomposition as used in the art. To start, an aqueous solution was made by combining Ca(NO₃)₂.4H₂O (0.06 mol/L), NaH₂PO₄ (0.06 mol/L), gelatin (1.2 g/L), and urea (0.12 mol/L) in deionized water (DiW) at room temperature. Once the reagents were completely dissolved, the contents were poured into a boiling flask, and the entire mixture was heated to 95° C. for 96 h. The HA nanowhiskers were then washed with DiW three times to remove the ammonia that results from the decomposition of urea. The synthesized HA whiskers have a thin layer of gelatin on their surface, which is essential for the particles to grow preferentially along the longitudinal axis, yielding a high aspect ratio. However, the presence of gelatin on the particles' surface increases particle agglomeration and can be detrimental to the bonding between the HA particles and PLA matrix in the final composite. For this reason, after being rinsed with DiW, the HA particles were stirred in a 0.5 M solution of acetic acid overnight, which is a concentration that is known to dissolve gelatin but leave HA unscathed. The HA was then washed three more times with DiW and dried for future use.

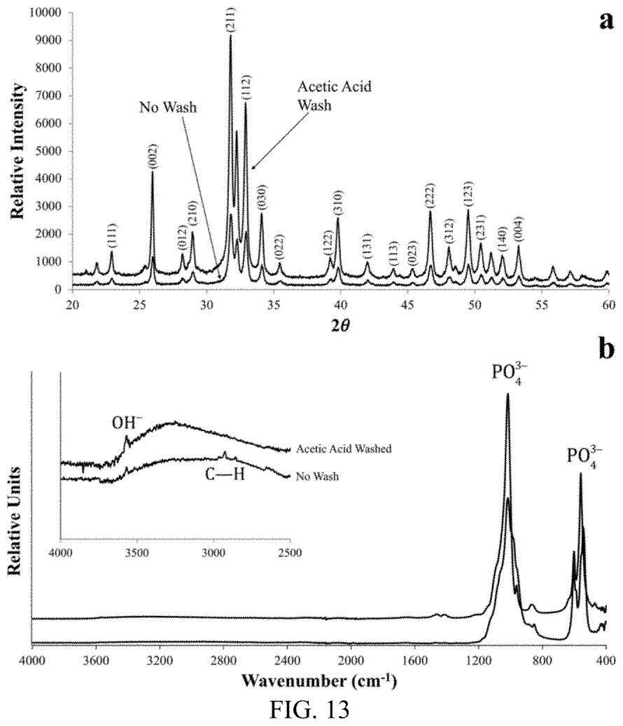

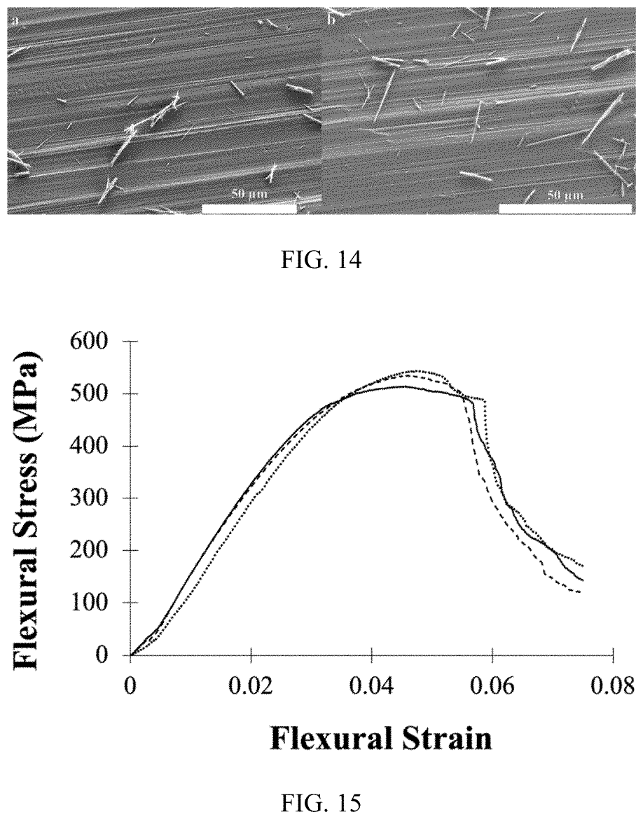

The FTIR and XRD spectra are shown in FIG. 13 a and b, respectively. The FTIR spectra show peaks that are characteristic of hydroxyapatite, where the peaks for the phosphate functional group (PO₄³⁻) are at 560 and 1000 cm⁻¹, the peaks at 1500 and 3400 cm⁻¹ correlate to H₂O, and the peak at 3500 cm⁻¹, which is difficult to see because of the water peak, represents the hydroxide group (OH⁻). Prior to the acetic acid wash, gelatin is observed at the peaks seen at 2825 and 2910 cm⁻¹ (C—H bonds) and the small peaks at 1350 cm⁻¹ (C—N bonds). The absence of these peaks in the spectra taken after the acetic acid wash proves the efficacy of the acetic acid at removing the gelatin coating. The XRD spectra in Supplemental FIG. 13b indicate the synthesized hydroxyapatite matches the pattern of standard HA ICDD 9-432. Both spectra also reveal that no other phase is present before or after the acetic acid washing, suggesting that the removal of gelatin does not coincide with a phase change of HA. Maintaining the HA phase is crucial because aside from substituted calcium phosphates (i.e. FHA), HA has superior mechanical properties and more desirable degradation properties when compared with other calcium phosphates. The FESEM micrographs in FIG. 14 show that the acetic acid treatment does not affect the morphology of the HA nanowhiskers, and the final particles have an average length, diameter, and aspect ratio of 6.8±4.0 μm, 0.22±0.07 μm, and 31.5±15.5, respectively.

### Example 6: Mechanical Properties

Two custom molds were fabricated. The first mold results in rectangular composite bars with a length and width of 55 mm and 12.7 mm, respectively. These rectangular bars were used to obtain flexural modulus values for the composites, which allows for a direct comparison to the modulus of natural bone and other fixation device materials. The second mold was designed based on currently used metal devices, which have a curvature so they contour to the bone. The radius of curvature of a sample metal fixation device was measured using a Gage Master GMX optical comparator. From this, a radius of curvature of 12.7 mm was incorporated into the compression mold design. The length and width of the slots in the mold were 60 mm and 10 mm, respectively, the latter of which was also based on current metal designs. When making the rectangular bars, the coated SF fibers were wrapped around the consolidation frame 125 times, and when making the curved bars, the coated SF fibers were wrapped around the consolidation frame 75 times. Before placing the consolidated fibers in the compression molds, the mold slots were sprayed with a demolding spray (WD-40 Specialist Dry Lube PTFE Spray) to guarantee the samples would not stick to the surfaces of the mold. The mold was then placed in a Carver press and heated to 170° C. prior to pressing the samples. The samples were pressed with a pressure up to 200 MPa, increasing by increments of 40 MPa. The pressure was held for 2 minutes at each increment, held for 15 minutes at 200 MPa, and finally the mold was cooled to room temperature. The frame was cut from the mold and then the mold was clamped down, and a hammer and chisel were used to shear the frayed ends of the composites off, leaving just dense, nonporous SF/HA/PLA composites. This was the final step of sample preparation for the rectangular composites, but to make the curved composites more realistic with respect to metal devices, holes were drilled into them. Special care was taken to drill clean holes. Following similar methods used to drill holes in carbon fiber laminates, a brad-point drill bit was used to drill the holes. The composite sample was then placed in a wooden mold to avoid fiber pushout, mounted in a drill press, and drilled slowly. A total of two holes were drilled in each sample, each with their center placed 10 mm from either end of the sample and both in the middle of the sample's y-axis. The resulting composite bars then underwent further analysis.

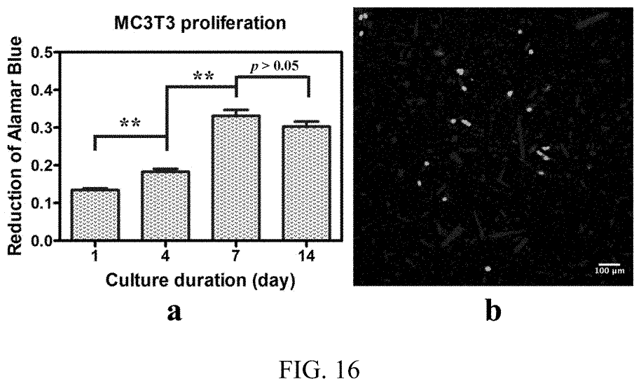

As shown by the TGA results (data not shown), rectangular and curved composite samples contained an average of 30.3±1.8 wt % and 30.1±1.6 wt %, respectively. Using the calculation outlined in the art, it was determined that this wt % correlated to an average volume fraction of 19.6 vol %. FIG. 15 shows the stress vs. strain curves for the rectangular samples tested via three-point bending in this study. This reveals that with 19.6 vol % HA the composite bars achieved a flexural modulus and strength of 21.1±0.45 GPa and 536±23 MPa, respectively. These curves also indicate that the samples fractured via fibrous failure, and no catastrophic failure was observed during the study.

### Example 7: In Vitro Cell Viability

To evaluate the cell viability of cells seeded on the composites, a ReadyProbe™ Blue/Green Cell Viability Imaging Kit (Invitrogen, USA) was used. Briefly, MC3T3 cells were seeded on the composites at a density of 1×10⁵ cells/well (n=2). After an incubation of 14 days, cell-attached samples were rinsed against PBS twice for 10 min and then stained in CCM containing the Kit for 30 min before imaging using Nikon A1R laser scanning confocal microscopy (LSCM). In this assay, the NucBlue™ Live reagent stains the nuclei of all cells while the NucGreen™ Dead regent stains only the nuclei of dead cells with compromised plasma membranes. To void the autofluorescence from the composite, a sample without cells was also stained and imaged accordingly as a negative control.

FIG. 16a shows the results from the alamarBlue cell proliferation assay, in which there is a statistically significant increase in the reduction of alamarBlue until day 7. This increase in alamarBlue reduction indicates there is an increase in cell proliferation during the first 7 days of the study. However, on day 14 there is no statistical change in cell proliferation when compared to day 7. FIG. 16 b shows the overall cell viability on the composites is good, with there being a live:dead cell ratio of 19:1.

The results from the cell viability assay show that cells attach well to the composite samples.

The use of the terms “a” and “an” and “the” and similar referents (especially in the context of the following claims) are to be construed to cover both the singular and the plural, unless otherwise indicated herein or clearly contradicted by context. The terms first, second, etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values is merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A load-bearing bone fixation composite, comprising
a polymer matrix,
a plurality of polymer fibers aligned along a common axis and disposed in the polymer matrix, wherein the polymer matrix binds the surface of the polymer fibers, and
a plurality of high aspect ratio nanorods coating at least a portion of each of the polymer fibers, wherein the long axis of at least a portion of the nanorods is aligned with the common axis, and wherein the high aspect nanorods have an aspect ratio of 10 or greater.

2. The composite of claim 1, wherein the polymer matrix comprises polylactic acid (PLA), poly(ε-caprolactone) (PCL), or a combination thereof.

3. The composite of claim 1, wherein the polymer matrix comprises about 5 to about 40% of the volume of the composite.

4. The composite of claim 1, wherein the polymer fibers comprise poly(L-lactic) acid (PLLA) fibers, silk fibroin fibers, polyglycolic acid (PGA) fibers, polydioxanone (PDO) fibers, or a combination thereof.

5. The composite of claim 1, wherein the polymer fibers comprise degummed Bombyx mori silkworm fibers.

6. The composite of claim 1, wherein the polymer fibers comprise about 30 to about 70% of the volume of the composite.

7. The composite of claim 1, wherein the nanorods comprise bioglass, calcium sulfate, calcium phosphate, or calcium silicate.

8. The composite of claim 1, wherein the nanorods comprise hydroxyapatite, ion-substituted apatite, carbonate hydroxyapatite, fluorinated hydroxyapatite, chlorinated hydroxyapatite, silicon-containing hydroxyapatite, tricalcinm phosphate, tetracalcium phosphate, monotite, dicalcium phosphate, dicalcium phosphate dihydrate, octacalciumn phosphate, calcium phosphate monohydrate, alpha-tricalcium phosphate, beta-tricalcium phosphate, amorphous calcium phosphate, biphasic calcium phosphate, calcium deficient hydroxyapatite, precipitated hydroxyapatite, and oxyapatite, or a combination thereof.

9. The composite of claim 1, wherein the nanorods comprise hydroxyapatite.

10. The composite of claim 1, wherein the nanorods comprise hydroxyapatite nanowhiskers.

11. The composite of claim 1, wherein the nanorods have an aspect ratio of greater than 30.

12. The composite of claim 1, wherein the nanorods comprise about 5 to about 30% of the volume of the composite.

13. The composite of claim 1, having a flexural modulus of 7 GPa to 21 GPa, and/or a flexural strength of 200-550 MPA measured according to ASTM D7264.

14. A bone fixation device comprising the composite of claim 1.

15. A method of bone fixation, comprising affixing the composite of claim 1 to a site of a load-bearing bone fracture, or maxillofacial bone fracture.

16. A method of making a load-bearing bone fixation composite, comprising
coating a plurality of polymer fibers with a suspension containing a polymer matrix, a plurality of high aspect ratio nanorods, and a solvent for the polymer matrix, wherein the polymer matrix binds the surface of the polymer fibers, and wherein the high aspect nanorods have an aspect ratio of 10 or greater,
optionally repeating the coating,
aligning the plurality of coated polymer fibers, and
consolidating the plurality of coated polymer fibers to provide the composite.

17. The method of claim 16, wherein aligning the plurality of coated polymer fibers comprises wrapping the plurality of coated polymer fibers on a frame.

18. The method of claim 17, wherein consolidating the plurality of coated polymer fibers comprises placing the frame in a mold and pressing the fibers under heat and pressure, wherein the press temperature melts the polymer matrix but not the polymer fibers.
